(19) United States
(12) Patent Application Publication
(10) Pub. No.: US 2006/0094115 A1
(43) Pub. Date: May 4, 2006

(54) Method for the production of cells with increased development potential

(76) Inventors: Ulf Rudiger Rapp, Wurzburg (DE); Bernd Neufeld, Koln (DE)

Correspondence Address: MAYER, FORTKORT & WILLIAMS, PC, 251 NORTH AVENUE WEST, 2ND FLOOR, WESTFIELD, NJ, 07090, US

(21) Appl. No.: 10/513,500

(22) PCT Filed: May 7, 2003
(86) PCT No.: PCT/DE03/01516

§ 371 Date: Oct. 3, 2005

(30) Foreign Application Priority Data
May 7, 2002 (DE) 102 20 480.2

(51) Int. Cl. C12N 15/87; C12N 5/08; A61K 35/30

(52) U.S. Cl. 435/455; 435/368; 424/93.7

(57) ABSTRACT

The invention relates to a method for producing cells having an increased development potential from cells, which are removed from an organism, wherein the removed cells are cultivated in vitro, and wherein a native nuclear non-activated MAPKAP kinase of the cultivated cell is activated or wherein an activated MAPKAP kinase is transported into the cell nucleus of the cells.

### FIELD OF THE INVENTION

The invention relates to a method for producing cells having an increased development potential, to cells obtainable by such a method, and to uses of such cells. Background of the invention and prior art.

During the embryonic and fetal phase, an organism is formed by differentiation of effector cells from stem cells. These embryonic or fetal stem cells have an increased development potential, compared to somatic cells from adult tissues. This is called the pluripotency of the embryonic or fetal stem cells, since they are capable to differentiate to all tissues, organs or cell types.

The production of embryonic stem cells for other purposes than for the in vitro fertilization is however a cause for very severe ethical concerns. For this reason, the search for alternative sources of pluripotent or totipotent cells is concentrated on the generation of a development potential of somatic cells being increased with respect to the natural state, in particular somatic stem cells from adult organisms. These research trends are encouraged by the finding that a transdifferentiation of somatic stem cells is possible, for instance neural stem cells can develop blood cells, whereas blood stem cells can produce brain and muscle cells. With regard thereto, reference is for instance made to the documents U.S. Pat. No. 6,087,168, U.S. Pat. No. 6,093,531 and U.S. Pat. No. 6,093,531. Increasing the plasticity of the somatic stem cells is therefore desirable, in order to permit, by means of somatic stem cells taken from an organism, treatment of the same and reimplantation, various organ repairs. Herein, the plasticity plays a special role, since with increased plasticity, such cells having a good availability (amount and/or easy removal), for instance hematopoietic stem cells, can be used as initial cells.

3 pK belongs to a kinase family (MAPKAP kinases), which are activated by one or more members of the family MAPK (mitogen-activated protein kinase). 3pK is also known as MAPKAP kinase 3 (MAPK activated protein kinase 3). Relatively well clarified are the mechanisms for the activation of 3pK by the mitogenic kinase cascade, and for instance reference is made to the documents G. Sithanadam et al.; Mol. Cell Biol. 16(3), 868-876, and S. Ludwig et al., Mol. Cell Biol. 16(12), 6687-6697. 3 pK can however also be activated by stress kinase cascades via p38 (S. Ludwig et al., Mol. Cell Biol. 16:6687-6697 (1996); M. M. McLaughlin et al., J. Biol. Chem. 271:8448-8492 (1996)), i.e. by induction of Rac (CDC42), for instance by heat shock or proapoptotic substances, such as TNF alpha. With respect thereto, for instance reference is made to the documents J. M. Kyriakis et al., Nature 369:199-211 (1994), J. Raingeaud et al., J. Biol. Chem. 279:7420-7426 (1995), A. Minden et al., Cell 81:1147-1157 (1995), S. Zhang et al., J. Biol. Chem. 279:23934-23936, J. Han et al., Science 265:808-811 (1994), S. Kumar et al. Biochem. Biophys. Res. Commun. 235:533-538 (1997), G. Wang et al., Cytogenet. Cell Genet. 78:50-55 (1997) and C. J. Lee et al., Nature 372:739-746 (1994).

Another MAPKAP kinase is MK2, which is primarily activated by p38 as a cellular response to stress, for instance by heat shock or cytokines such as TNF alpha (J. Rouse et al., Cell 78:1027-1037 (1994); K. Engel et al., J. Cell Biochem. 57:321-330 (1995)). 3pK and MK2 have on the amino acid level a sequence homology of 75%. Both are disposed in the not activated state in the cell nucleus and leave it after activation or phoshorylation. Further homologs are MAPKAP-K1, RSK2, RSK3, MK4 and PRAK.

Little is known about the physiological substrates with regard to the physiological functions of MAPKAP kinases, such as 3pK or MK2.

The differential expression of homeotic genes determines the formation of different body parts along the longitudinal axis of an organism. In the early embryonic phase, homeotic genes are activated in the correct regions by transcription factors. These factors are however soon after that not active anymore, and the accurate expression of homeotic genes in the course of the further development of the organism is controlled, among other factors, by proteins of the so-called polycomb group (Pc-G). Pc-G proteins hold genes repressed, which are not expressed anymore after a certain development stage. This property is also transferred to the daughter cells. They constitute part of a cellular memory system, are thus part of an epigenetic expression control. It is assumed that the Pc-G proteins generate a chromatin configuration, which is in accessible for transcription factors, and thus inhibit the transcription of homeotic genes. With regard to the function of the Pc-G proteins, reference is in particular made to the following survey article: J. J. L. Jacobs et al., Cell & Development Biology, Vol. 10, 1999, pp. 227-235. Members of the Pc-G family are, among others, BMI1 and HPH1 or HPH2 (human polyhomeotic).

Generally can be said that some knowledge about the effective mechanisms of the Pc-G proteins has already been gained. However, in contrast thereto, little is known about how the Pc-G proteins themselves are regulated.

### TECHNICAL OBJECT OF THE INVENTION

It is the technical object of the invention to provide a method for increasing the plasticity of cells.

### BASICS OF THE INVENTION

For achieving this technical object, the invention teaches a method for producing cells having an increased development potential from cells, wherein the removed cells are cultivated in vitro, and wherein a native nuclear non-activated MAPKAP kinase of the cultivated cells is activated or wherein an activated MAPKAP kinase is transported into the cell nucleus. The term nuclear designates a localization of the MAPKAP kinase in the cell nucleus, in particular in the chromatin structure. A MAPKAP kinase is activatable, if it can be phosphorylated with the consequence that the phosphorylated MAPKAP kinase can phosphorylate its substrates and is exported from the cell nucleus. For the purpose of the invention, an activation of the said MAPKAP kinase may however also be performed immediately in vivo, and then the cells according to the invention are generated immediately in vivo.

The invention is based on the finding that nuclear MAPKAP kinases bind with HPH2 as well as with BMI1. The invention is based on the further finding that the activated phosphorylated MAPKAP kinase in turn phosphorylates BMI1 still prior to the export from the cell nucleus, the BMI1 thus also being mobilized, and thereby at last a locus repressed by the Pc-G complex becoming accessible and transcribed. Hereby genes, which normally are inactive after a certain development stage, are reactivated, thus the cells being returned to a state being similar to early embryonic stem cells, i.e. the plasticity of the used cells is increased. In principle, the invention can be used for all cell types, particularly suitable are however somatic stem cells.

In detail, the following findings are used. 3pK is in vitro a BMI1 kinase and develops in an artificial regression assay a similarly repressing effect as other Pc-G proteins. 3pK binds to presumed full-length HPH2 and has the highest affinity to a 73 amino acids long C-terminal fragment of HPH2, which comprises the HDII/SEP domain. This region is part of the HPH2 dimerization domain, i.e. it comprises the alpha-helical HDII/SEP domain required for the hetero/homodimerization as well as for the BMI1 binding and overlaps with the domain for 3pK binding. The stronger binding to the said fragment compared to the full length may be caused by a folding of full length HPH2 over the HDII/SEP domain. The state of the phosphorylation of HPH2 is not clear, and a phosphorylation of the presumed full length HPH2 by 3pK could not be observed in vitro. Phosphorylation is however not excluded, since a mouse homolog of the HPH2 (mPh2, NCBI accession U81491) with a long N-terminal extension contains three potential 3 pK phosphorylation sites. Since HPH1 and mouse mPh1 proteins have the same length, it can be assumed that HPH2, too, has the same length as mPh2.

The above findings are also supported by that BMI1 coprecipitates with 3pK. 3pK is a kinase, which phosphorylates with BMI1. This plays an important role, since on the one hand the dissociation of Pc-G complexes and the BMI1 phosphorylation are connected with each other, and on the other hand hypophosphorylated BMI1 is specifically held in the chromatin-associated protein fraction from the cell nucleus, whereas phosphorylated BMI1 is not chromatin-bound. In summary it was found that 3pK forms part of the Pc-G complex and is a BMI1 kinase. 3pK will consequently regulate the phosphorylation-dependent Pc-G complex/chromatin interaction, by binding to HPH2, thus 3pK being made available to BMI1 in the Pc-G complex, with the consequence of its phosphorylation and release of the complex from the chromatin. Similar situations were found in the case of MK2.

Interestingly, the phosphorylation state of the 3pK does not seem to be relevant for the binding to HPH2. Comparison tests with 3pK variants mutated at known phosphorylation sites of the 3pK (T313E, T313A, TT201/313EE) did not shown any changes of the binding to HPH2. Further, 3pK also precipitates after treatment of the cells with arsenite (a strong agent for the induction of the 3pK phosphorylation and activation) with Pc-G complexes. By the way, coprecipitation takes also place with endogenous 3pK and Pc-G complexes.

The invention further relates to cells obtainable by a method according to the invention and to the use of cells according to the invention for producing a pharmaceutical composition in particular for the treatment of degenerative nerve diseases. For such diseases, the increase of the plasticity secures a settlement of the target tissue and a differentiation of the introduced cells according to the invention to the desired cell type.

A particular aspect of the findings according to the invention of independent importance is also the use of a substance promoting the expression or activation of a MAPKAP kinase, in particular 3pK, for producing a pharmaceutical composition for the prophylaxis or treatment of cancer diseases. Alternatively, by genetic measures the tumor cells can be brought to the expression of activated 3pK. For this aspect, the fact is used that BMI1 has oncogenic functions, and that the modulation thereof can thus control the proliferation of cells. It is known, for instance, that reduced proliferation may be based on an overexpression of the tumor suppressors p16 and p19ARF, which are both coded by the INK4a tumor suppressor locus (see J. J. Jacobs et al., Nature 397:164-168 (1999)). A release of this locus in tumor cells would consequently induce the said tumor suppressors with the result of the inhibition or reduction of the proliferation being increased because of the disease. Besides, the above and below explanations apply in an analogous manner. Substances, which induce or activate 3pK in tumor cells, are consequently suitable for producing pharmaceutical compositions for the treatment of cancer. It is recommended either to locally apply the substances or to couple them to tumor cell-specific substances, such as interaction partners of tumor markers or ligands of tumor cell-specific receptors.

### PREFERRED EMBODIMENTS OF THE INVENTION

The MAPKAP kinase may be 3pK, MNK1, MNK2, MSK1, MK2, MK4 or PRAK, preferably 3pK. In the case of the activation of native MAPKAP kinase, it is preferably a wild-type MAPKAP kinase.

Activated MAPKAP kinase can be induced in various ways in the cell nucleus. It is for instance possible that the cells are transformed to overexpression of an activated MAPKAP kinase. The activation by induction of p38 is also possible, and the induction of p38 can in turn take place by induction of Rac and/or Ras.

The activation of 3pK can in detail take place by induction of the mitogenic kinase cascade, in particular induction of Raf, MEK and/or ERK, for instance by incubation with a serum growth factor or with TPA. The activation can however also take place by induction of a stress kinase cascade, for instance I) by treatment of the cells with conditions inducing the stress kinase cascade, in particular a heat shock, osmolarity shock or UV, or ii) by incubation in a physiologically effective dose with at least one substance inducing the stress kinase cascade, in particular cytokines such as IL-1, TNF-alpha, anisomycin, arsenite and/or alkylating agents.

Of special importance is the observation that the presence of B-raf in cells, naturally existing or artificially induced, is important for the increase of the development potential. It was observed, e.g., that with absence of B-raf a generation or an increase of the plasticity of neuronal stem cells in the mouse will not take place. Therefore, a preferred aspect of the invention is the generation or induction and/or acquisition of B-raf in target cells, the development potential of which is to be increased.

It is preferred that the used cells are somatic stem cells, for instance hematopoietic or neural stem cells. For reasons of the better immune tolerability, the used cells should be autologous, whereby with the administration or reimplantation undesired immune reactions are prevented.

In the following, the invention is explained in more detail, based on examples and tests.

Methods:

Plasmid Construction:

Human PKRSPA-3pK, which carries the gene under the control of the Rous sarcoma virus (RSV), pEBG-3pK, which expresses the gene as a GST fusion protein under the control of the human EF1a promoter, and the mutation of the lysine in the region of the presumed ATP binding region of the 3pK (3pK K73M) are known in the art from the document S. Ludwig et al. (see above). Another mutation at 73 or derivatization making 3pK inactive can also be used. PcDNA3HA-3pK was produced by insertion of a NheI-SpeI fragment of pPC97-3pK in pcDNA3HA. pPCH-3pK K73M was obtained by insertion of the SalI-NotI fragment of pPC97-3pK into pPCH (obtained from C. Hagemann, MSZ, Wuerzburg), which is a pPC97 derivative, wherein the LEU nutrient cassette is replaced by the TRP marker of pPC86 (P. M. Chevray et al., Proc. Natl. Acad. Sci. USA 89(13), 1992, 5789-5793). The BamHI-BamHI insert of pGEXKG-3pK was ligated into the yeast two-hybrid vector pAS2.1 (Clontech), in order to obtain pAS2.1-3pK K73M. pMT2SM-HA-bmi (mouse), ppuro GAL4 DB-Bmi1 and ppuro GAL4 DB were obtained from M. v. Lohuizen (Amsterdam). By cutting pGEXKG-3pK with EcoRI and subsequent insertion of this fragment into ppuro GAL4 DB, ppuro GAL4 DB 3pK was cloned. pBEVY-GU-Xbmi was obtained by ligation of the EcoRI fragment of pPC97-Xbmi (obtained from A. Otte, Amsterdam) in pBEVY-GU (obtained from Ch. A. Miller, New Orleans). pGAD10-HPH2 (137-432 aa), in the following called pGAD10-HPH2 (C-295 aa), and pGAD10-HPH2 (432 aa) can be obtained from A. Otte and are described in M. J. Gunster et al., Mol. Cell. Biol. 17(4), 2326-2335.

Yeast Two-Hybrid System:

The yeast two-hybrid screens were performed under utilization of a human heart MATCHMAKER cDNA library, which is cloned into the yeast two-hybrid vector pGAD10 (Clontech). In the first screen the kinase-inactive 3pK K73M in the single copy plasmid PPCH was used. The yeast strain CG-1945 was manipulated according to the MATCHMAKER Library User Manual (PT1020-1, Clontech), and the sequential transformation protocol was used. Positive clones were identified by growth on SD/−TRP/−LEU/−HIS plates and activity determination of the lacz reporter gene in filter assays. A DNA sequence analysis showed that the two cDNA library plasmids independently isolated contained the same 879 bp of HPH2 coding a 73 aa (220 bp) and a part of the 3UTR (659 bp). An independent screen of the same two-hybrid libraries under utilization of pAS2.1-3pK K73M as a bait and the yeast strain Y190, which comprises a higher lacZ reporter gene expression, resulted in 128 β-gal positive clones grown on SD/−TRP/−LEU/−HIS/+25 mM 3-AT (3-amino-1,2,4-triazol), of which in turn 17 coded C-terminal fragments of HPH2: five clones contained the fragment of the first screen, four cDNAs coded 145, three coded 198 and five coded 209 C-terminal amino acids. Direct two-hybrid tests were also made with these interaction partners and pAS2.1-3pK K73M, corresponding to standard protocols. The liquid culture β-galactosidase assay with ONPG (o-nitrophenyl-β-D-galactopyranoside) as a substrate was performed under utilization of the strain Y190 according to the MATCHMAKER instructions of use. In order to reduce the variation, five different transformants of every cotransfection were investigated. pAS2.1-3pK K73M was cotransformed as a bait with the following six different (length) constructs: GAL4 AD-HPH2 (C-73aa), GAL4 AD-HPH2 (C-145aa), GAL4 AD-HPH2 (C-198aa), GAL4 AD-HPH2 (C-209aa), GAL4 AD-HPH2 (C-295aa), and GAL4 AD-HPH2 (C-432aa). In another series of experiments, pAS2.1-3pK K73M and pGAD10-HPH2 (C-73aa) were coexpressed with a third yeast expression vector, either pBEVY-GU without insert or PbEVY-GU-Xbmi1, and cultivated on SD/−TRP/−LEU/−URA plates and then subjected to the liquid culture β-galactosidase assay.

Cell Cultures and Antibodies:

The human embryonic kidney cell line HEK293, which was used for immunocomplex kinase assays and coimmunoprecipitation experiments, was cultivated in Dulbecco's Modified Eagle medium (DMEM), supplemented with 10% (v/v) FCS (heatinactivated at 56° C. for 30 min), at 37° C. in humid air with 6% CO₂. The human osteosarcoma cell line U20S GAL4-TKluc, which is stably transfected with a TK luciferase reporter plasmid with five GAL4 binding sites above the TK promoter and was used for the repression assay, was cultivated in DMEM with 10% FCS (v/v). Polyclonal rabbit antiserum against bacterially expressed 3pK was obtained according to G. Sithanandam (see above). Anti-glutathione S-transferase (GST) antisera were obtained from rabbits, which were immunized with bacterially expressed and purified GST. 1:750 dilutions of both sera were used for immunoblots. The monoclonal anti-HA tags (12CA5) were used in a concentration of 1 μg/ml for Western blots.

Transfection and 3pK Activation:

For the transfection of both cell lines HEK293 and U-2OS, 5×10ˆcells were sown in a 10 cm dish and cultivated for 24 h in DMEM with 10% FCS prior to the transfection. The transfections were performed by means of the calcium phosphate coprecipitation method under utilization of 5-10 μg DNA for HEK293 or 15 μg DNA for U-2OS corresponding to a modified stratagene protocol (according to S. Ludwig et al., see above). HEK293 cells were starved 48 h before the transfection in DMEM with 0.3% FCS. For the activation with 3pK, HEK293 cells stimulated with 0.5 mM sodium metaarsenite 30 min before the harvest or with 20% FCS in combination with 100 ng tetradecanoylphorbol acetate (TPA) per ml 60 min before the harvest.

Immunoprecipitation and Immunoblots:

Transfected HEK293 cells were lysated in Triton lysis buffer (TLB, 20 mM Tris (pH 7.4), 50 mM sodium β-glycerophosphate, 20 mM sodium pyrophosphate, 137 mM NaCl, 10% (v/v) glycerol, 1% (v/v) Triton X-100, 2 mM EDTA, 1 mM Pefabloc, 1 mM sodium orthovanadate, 5 mM benzamidine, 5 mg per ml apritinine, 5 mg per ml leupeptin) on ice for 10 min. Cell solids were removed by centrifugation at 15,000 rpm for 10 min. Supernatants were incubated with various antisera for 2 h at 4° C. Untagged 3pK was immunoprecipitated with anti-3pK antiserum. Tagged variants of the proteins were immunopurified with corresponding anti-tag antibodies. The immunocomplexes were precipitated with 25 μl protein A agarose and extensively washed either with TLB or RIPA (1M NaCl) for coimmunoprecipitation or immunocomplex kinase assays. For the detection of the proteins in Western blots, the immunocomplexes were suspended in electrophoresis sample buffer and heated for 3 min at 100° C. After SDS PAGE, gels were electro-blotted on nitrocellulose membrane (Schleicher & Schuell) and forwarded to immune detection under utilization of the corresponding primary antibodies. Proteins were visualized by means of horse radish peroxidase-conjugated protein A agarose (Amersham) and a standardized reinforced chemiluminescence reaction (Amersham).

Immunocomplex Kinase Assay with 3pK:

Separately immunoprecipitated GST-3pK and HA-BMI1 were combined and washed twice with a high NaCl concentration (25 mM Tris (pH 8), 1 M NaCl, 10% (v/v) glycerol, 0.1% SDS, 0.5% sodium deoxycholate, 1% NP40, 2 mM EDTA (pH 8), 1 mM Pefabloc, 1 mM sodium orthovanadate, 5 mM benzamidine, 5 mg per ml aproptin, 5 mg per ml leupeptin) and specific kinase buffer (10 mM MgCl2, 25 mM β-glycerophosphate, 25 HEPES (pH 7.5), 5 mM benzamidine, 0.5 mM dithiothreitol, 1 mM sodium vanadate). The kinase assays were performed at 30° C. in the same buffers, supplemented with 5 mCi [gamma-32-P]-ATP, 0.1 mM ATP, and the reaction was terminated after 30 min by addition of Laemmli buffer and 3 min at 100° C. After gel electrophoresis and blotting on nitrocellulose membrane, the BMI1 phosphorylation was analyzed by means of an X-ray film (Amersham).

Repression Assay:

These experiments were substantially performed according to document M. J. Alkema et al., J. Mol. Biol. 173, 1997, 993-1003. U2OS were cultivated in 10 cm dishes up to 40-60% cofluency and transiently transfected under utilization of calcium phosphate. Every 10 cm dish was transfected with either 15 μg ppuro GAL4 DB, ppuro GAL4 DB-BMI1, ppuro GAL4 DB-3pK or PKRSPA and 2 μg of a β-GAL reporter gene. After 20-24 h, the transfection medium was replaced after several washings with PBS for removing precipitates by DMEM with 10% FCS. After 8-10 hours, this medium was replaced by DMEM with 10% FCS and 10 μg/ml puromycin, where the cells were cultivated for another 38-62 h for removal of all non-transfected cells. Cells, which were transfected with the not puromycin-resistant vector pKRSPA, served as a control for the complete elimination. The remaining cells were then cultivated for 6 h in DMEM with 10% FCS, whereafter the cells were investigated for luciferase and β-gal activity. U2OS were harvested in 100 ml lysis buffer (50 mM Na-2(N-morpholino)ethane sulfonic acid, pH 7.8, 50 mM Tris HCl, pH 7.8, 10 mM dithiothreitol, 2% Triton X-100). The untreated cell lysates were purified by centrifugation, and 50 ml of the prepurified cell extract were added to 50 ml luciferase assay buffer (125 mM Na-2 (N-morpholino)ethane sulfonic acid, pH 7.8, 125 mM Tris HCl, pH 7.8, 25 mM magnesium acetate, 2 mg/ml ATP). Immediately after injection of 50 ml 1 mM D-Luciferins (Applichem) to every sample, the luminescence was measured for 5 s in a luminometer (MicroLumat LB96P, EG&G Berthold). The β-galactosidase assay was performed with 20 μl cell lysate according to a standard protocol (J. Sambrook et al., Molecular Cloning: A Laboratory Manual, 2^{nd} Ed. 1989, Cold Spring Harbor Laboratory Press, New York). The relation of the background-corrected luciferase values and β-gal values of independently transfected dishes were used for correction of the transfection effectivity and referred to the empty vector-transfected controls. All transfections were performed at least twice, and standard deviations were calculated.

Results:

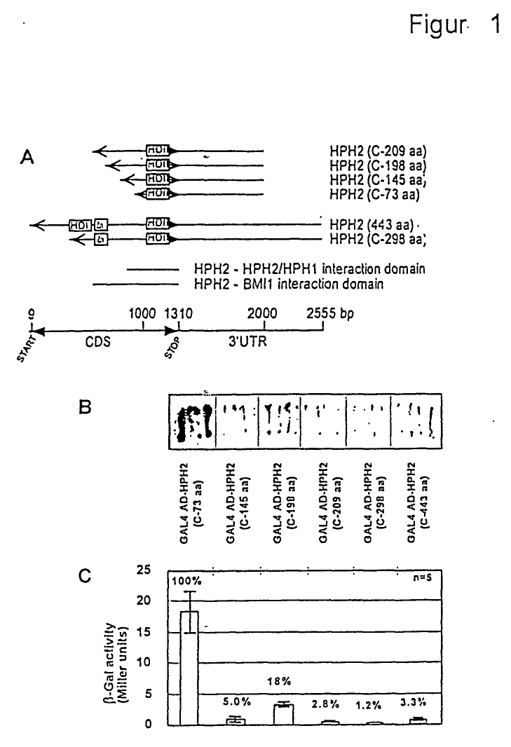

FIG. 1 shows as a result that 3pK binds in vivo in the yeast two-hybrid system to the C-terminal part, including the homology domain II (HD II), of HPH2. FIG. 1A is a diagrammatic representation of the HPH2 clones of different lengths interacting with 3pK. Interestingly, the domain (104 C-terminal amino acids) required for the homodimerization as well as for the heterodimerization with HPH2 or HPH1, as well as the binding domain for BMI1 (295 C-terminal amino acids) overlap with the interaction domain for 3pK binding. The minimum 3pK binding domain of HPH2 (73 C-terminal amino acids) comprises a 67 amino acids long homology domain II (HDII, called SAM or SEP), which is identical or similar to corresponding domains of other polyhomeotic proteins (e.g. Mph2, Mph1/Rae-28 or HPHII). The strongest interaction 3pK/HPH2 was found with the smallest HPH2 fragment, as can be seen from the x-gal test (FIG. 1B) and the quantitative two-hybrid test with ONPG as a substrate (FIG. 1C).

In the test of FIG. 1B, Y190 yeast was cotransformed with pAS2.1/3pK K73M and the cited pGAD10/HPH2 constructs and cultivated on SD/−TRP/−Leu plates. The obtained colonies were spread on filters, which were placed on SD/−TRP/−LEU/−HIS, and cultivated for 12-24 h at 30° C., before the β-gal assay was performed. For the quantification of the binding affinities, a liquid culture β-galactosidase assay with ONPG as a substrate was performed. The activity for 3pK-HPH2 (C73 aa) was set to 100% for comparison purposes.

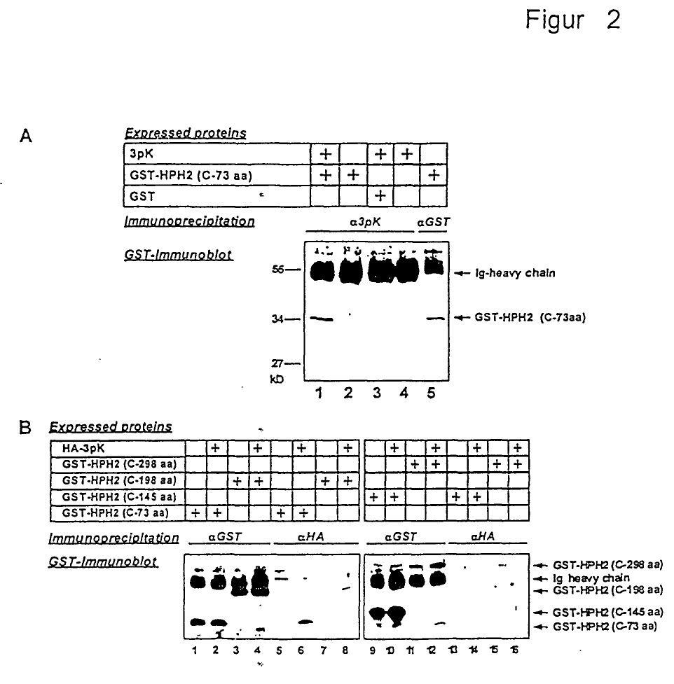

FIG. 2 shows that 3pK interacts in vivo with HPH2, and that also in the case of mammalian cells. 3pK will be part of the polycomb complex, which comprises HPH2. For the analysis, whether 3pK is present in a Pc-G together with HPH2, coimmunoprecipitation experiments were made. In a first series of experiments, HEK293 cells were cotransfected with GST tagged HPH2 (C-73 aa) and 3pK wt. As a positive control, polyclonal anti-GST serum was used for the immunoprecipitation and immunoblotting of GST-HPH2 (C-73 aa) (FIG. 2A, track 5). 3pK was precipitated with a 3pK-specific antiserum and coimmunoprecipitated GST-HPH2 (C-73 aa) was detected in Western blots with the anti-GST antiserum (FIG. 2A). From track 1 can be seen that GST-HPH2 (C-73 aa) precipitates with 3pK, whereas no coprecipitation is found in the case of the control samples with GST alone, or in absence of 3pK or GST-HPH2 (C-73 aa) (tracks 2 to 4).

In the tests of 2B, GST-HPH2 fragments of different lengths (as specified) were cotransfected with a HA-tagged variant of 3pK. Immunoprecipitation and immunoblotting with anti-GST antiserum confirms the expression of the various GST-HPH fusion proteins (tracks 1 to 4 and 9 to 12). Coimmunoprecipitation with HA-3pK under utilization of HA mab was observed in the case of GST-HPH2 (C-73 aa), track 6, and GST-HPH2 (C-198 aa), track 8. (C-145 aa) and (C-295) were not observed, presumably because of not sufficient binding affinity. This also confirms the binding strengths according to FIG. 1C.

The bands at 55 kDa are the result of a cross reaction with heavy chains of the antibodies in the samples and have nothing to do with the above considerations. In detail, the following was made. HEK 293 cells were transfected with the cited plasmids. 3pK was expressed either untagged by an RSV promoter (FIG. 2A) or as an HA-tagged version by a CMV promoter (FIG. 2B). The proteins were immunoprecipitated, as mentioned, either with anti-3pK (A), anti-GST (A, B) or Anti-HA (12CA5) (B) antibodies, washed twice in TLB buffer, separated on SDS-PAGE gels (10%) and blotted in nitrocellulose filter. Immunoblots were sampled with the respective antiserum and detected with ECL.

In tests not shown in the drawings, it was investigated whether 3pK is associated with chromatin. Differential nucleus extracts of cells, which overexpress human 3pK constructs, show that part of the wild-type 3pK only is associated with chromatin. In contrast, constitutively active of kinase-inactive 3pK mutants did not associate or associated to a clearly lower extent with chromatin, in agreement with other investigations, according to which such mutants are mainly present in the cytoplasm.

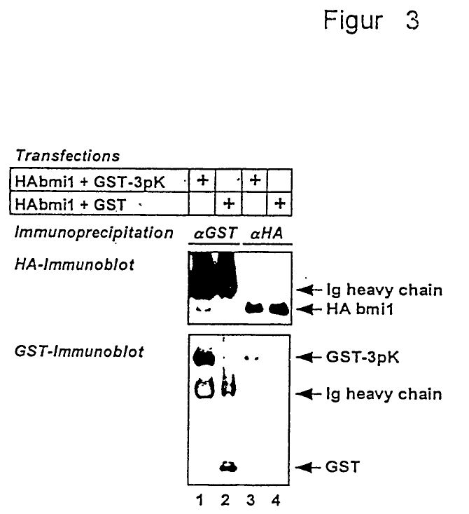

In FIG. 3 are shown results, which confirm an in vivo interaction between 3pK and BMI1. In detail, the following was made. The cells were transfected with HA-BMI1 or pEBG-3pK DNA. The immunoprecipitation was performed either with anti-GST or with Anti-HA (12CA5) antibodies, followed by washing twice with TLB buffer. The proteins were then separated on SDS-PAGE gels (10%) and blotted in nitrocellulose filter. Immunoblots were sampled with the respective antiserum and detected with ECL. The bands at 55 kDa have the above reason.

It can be seen that HA-BMI1 is specifically coprecipitated with GST-3pK (track 1) and vice versa (track 3). The tracks 2 and 4 do not show any interactions with HA-BMI1 or GST alone.

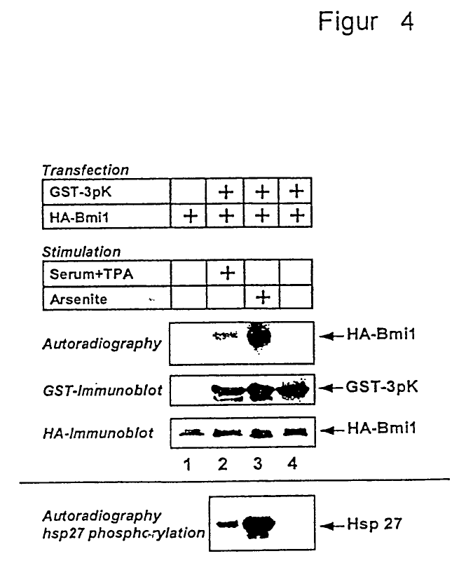

FIG. 4 shows that 3pK phosphorylates BMI1. HEK 293 cells were transfected with HA-BMI1 or pEBG-3pK DNA. The kinase was activated by stimulation of the cells with arsenite or serum/TPA for 60 min. Substrate and kinase were immunoprecipitated with anti-HA (12CA5) or anti-GST antibodies. Immunocomplexes were combined, washed twice under stringent conditions with RIPA buffer containing 1 mM NaCl and subjected to an in vitro kinase reaction in presence of [gamma32P]AP. The tracks 2 and 3 of the upper panel show that with activated 3pK, a strong phoshorylation takes place. However, there is no or only little phoshorylation in absence of 3pK or with lacking activation (tracks 1 and 4). The lower panel shows a positive control reaction under utilization of Hsp27 as a substrate for immunoprecipitated 3pK. This was found in HeLa and 293T cells, but also in primary human TIG3 fibroblasts, whereby is shown that the invention is not limited to certain cell types nor bound to specific cellular phenotypes.

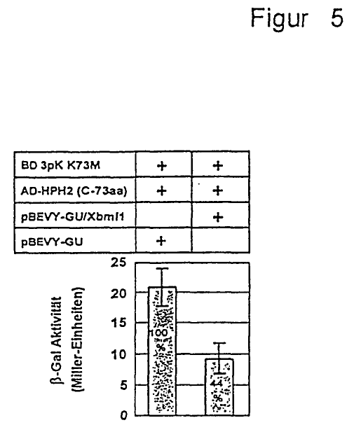

FIG. 5 shows that BMI1 weakens the interaction 3pK/HPH2. For the tests, an assay similar to the one of FIG. 1C was performed. In addition to the transformation of pAS2.1/3pK K73M and pGAD10/HPH2 (C-73 aa), either pBEVY-GU or pGEVY-GU/XBMI1 was cotransformed. XBMI1 (Xenopus BMI1) is by 90% identical and by 95% similar to human and mouse BMI1Da an ONPG β-galactosidase assay by 6 orders less sensitive than with X-gal, only the (C-73 aa) fragment was used. Every bar represents five parallel experiments, and the shown data are representative for five independent assays. It can be seen that XBMI1 reduces the binding strength from 100% to 44%.

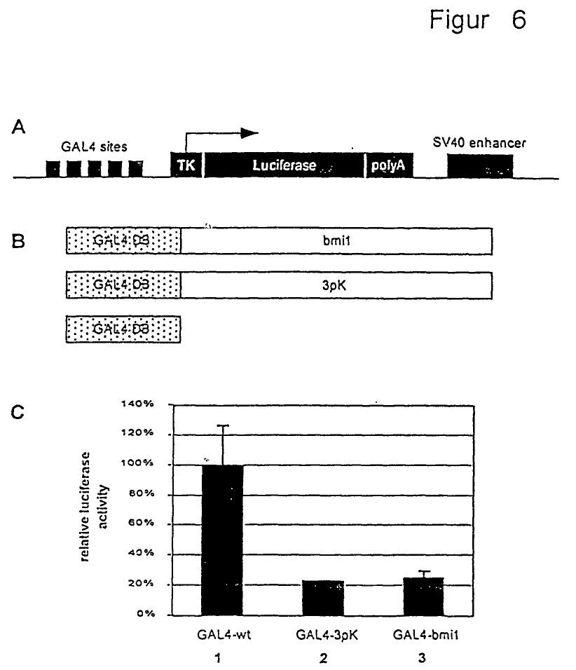

In FIG. 6 are shown the results of a repression assay. FIG. 6A shows a diagrammatic representation of the stably integrated luciferase reporter construct. FIG. 6B shows the used fusion proteins with the GAL4 DNA binding domain (Gal4 DB). For the results of FIG. 6C, the procedure was according to the statements under “Methods”.

As a positive control, the expression of Gal4 DB-BMI1 was used, and precisely the 4-fold inhibition, as reported by Alkema et al. (see above), was found. The same result was however also found for Gal4 DB-3pK, which confirms that non-activated and Pc-G-associated 3pK is capable to recruit Pc-G proteins, such as BMI1 or HPH2, to a chromatin-embedded promoter, where an inhibition complex is formed.

1. A method for in vitro production of cells with an increased development potential from cells, which have been removed from an organism, wherein the removed cells are cultivated in vitro, and wherein a native nuclear non-activated MAPKAP kinase of the cultivated cells is activated or wherein an activated MAPKAP kinase is transported into the cell nucleus of the cells.

2. A method according to claim 1, wherein the MAPKAP kinase is 3pK, MNK1, MNK2, MSK1, MK2, MK4 or PRAK, and preferably is a wild-type MAPKAP kinase.

3. A method according to claim 1 or 2, wherein the cells are transformed for overexpression of an activated MAPKAP kinase.

4. A method according to one of claims 1 to 3, wherein the activation takes place by induction of p38.

5. A method according to claim 4, wherein the induction of p38 takes place by induction of Rac and/or Ras.

6. A method according to one of claims 1 to 4, wherein the activation of 3pK takes place by induction of the mitogenic kinase cascade, in particular induction of Raf, MEK and/or ERK.

7. A method according to claim 6, wherein the cells are incubated with a serum growth factor or with TPA.

8. A method according to one of claims 1 to 5, wherein the activation takes place by induction of a stress kinase cascade.

9. A method according to claim 8, wherein the activation takes place i) by treatment of the cells with conditions inducing the stress kinase cascade, in particular a heat shock, osmolarity shock or UV, or ii) by incubation in a physiologically effective dose with at least one substance inducing the stress kinase cascade, in particular cytokines such as IL-1, TNF-alpha, anisomycin, arsenite and/or alkylating agents.

10. A method according to one of claims 1 to 9, wherein the cells are somatic stem cells.

11. A method according to one of claims 1 to 10, wherein the cells are removed from a non-embryonic human organism.

12. The cells obtainable by a method according to one of claims 1 to 11.

13. the use of cells according to claim 12 for producing a pharmaceutical composition, in particular for the treatment of degenerative nerve diseases.

14. The use according to claim 13, wherein the cells are autologous.

15. A method for the treatment of degenerative nerve diseases, wherein cells according to claim 12 are administered in a suitable galenic preparation to a diseased organism, or wherein a substance activating a MAPKAP kinase is administered in a suitable galenic preparation to a diseased organism.

16. The use of a substance activating a MAPKAP kinase for producing a pharmaceutical composition for the treatment of degenerative nerve diseases.
